(19) United States
(12) Patent Application Publication
(10) Pub. No.: US 2005/0186169 A1
(43) Pub. Date: Aug. 25, 2005

(54) Process for treating skin with a tetrapolymer

(75) Inventor: Yael Charbit, Maisons-Alfort (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/046,925

(22) Filed: Feb. 1, 2005

Related U.S. Application Data
(60) Provisional application No. 60/554,912, filed on Mar. 22, 2004.

(30) Foreign Application Priority Data
Feb. 23, 2004 (FR) 0450333

(51) Int. Cl. A61K 7/06; A61K 31/74

(52) U.S. Cl. 424/70.16

(57) ABSTRACT

The present invention relates to a treatment process for human skin, comprising the topical application to said skin of a composition comprising, in a physiologically acceptable medium, a tetrapolymer of methacrylic acid, methyl methacrylate, butyl acrylate and C₁₆-C₂₀ alkyl (meth)acrylates, said composition being devoid of sunscreen agents. The invention likewise relates to the use of a tetrapolymer of methacrylic acid, methyl methacrylate, butyl acrylate and C₁₆-C₂₀ alkyl (meth)acrylates.

### REFERENCE TO PRIOR APPLICATIONS

This application claims priority to U.S. provisional application 60/554,912 filed Mar. 22, 2004, and to French patent application 0450333 filed Feb. 23, 2004, both incorporated herein by reference.

### FIELD OF THE INVENTION

The present invention relates to a process, preferably a cosmetic treatment process, for human skin, comprising the topical application to said skin of a composition comprising, in a physiologically acceptable medium, at least one tetrapolymer of methacrylic acid, methyl methacrylate, butyl acrylate and C₁₆-C₂₀ alkyl (meth)acrylates, said composition preferably being devoid of sunscreen agents. The invention is particularly suited towards the treatment of wrinkled skin, and the signs of tiredness on the skin.

The invention likewise relates to the use of at least one tetrapolymer of methacrylic acid, methyl methacrylate, butyl acrylate and C₁₆-C₂₀ alkyl (meth)acrylates as a tightening agent for human skin.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

A current trend among women and even men is to appear young for as long as possible, and consequently such persons seek to disguise the signs of skin ageing, which are manifested in particular in wrinkles and fine lines. In this regard, advertising and fashion exhibit products which are intended to retain for as long as possible skin which is radiant and has no wrinkles, the signs of young skin, particularly since physical appearance has an effect on the psyche and/or on morale.

Wrinkles and fine lines have to date been treated using cosmetic products containing actives which act on the skin, for example by moisturizing it or by enhancing its cellular regeneration or else by promoting the synthesis, or preventing the degradation, of the elastic fibres which make up the cutaneous tissue.

These products, however, have the drawback of being effective for treating wrinkles only after a certain application time. More and more, however, the desire is to have anti-wrinkle products which have an immediate effect.

To this end, a number of years ago, agents were proposed which have a tightening effect, which immediately after their application smooth out wrinkles and fine lines and contribute to attenuating the signs of tiredness. These compounds act by forming a film which, on drying, causes the stratum corneum—the superficial horny layer of the epidermis—to retract.

Among these tightening agents plant proteins have been proposed for use. These proteins, however, give rise to formulation problems (loss of viscosity, stability problems) and are not always very cosmetic, in the sense that they endow the compositions comprising them with a sticky feel and even produce a disagreeable odour and/or colour.

Application EP-0 944 381, furthermore, proposed the use as tightening agents of latices composed of acrylic homopolymers and copolymers. Not all of these polymers, however, allow stable cosmetic compositions to be realized, especially when these compositions are formulated as emulsions. Constricting rules of formulation must be respected in this case. It has discovered, moreover, that the presence of a surfactant, which is generally necessary for stabilizing emulsions, has an adverse effect on the tightening power exerted by these polymers.

There is therefore still a need to have compounds which have tightening effects at least as great as those of the prior art, which are easy to formulate as stable compositions and which exhibit satisfactory cosmetic properties, so making these compositions pleasant to use.

### DETAILED DESCRIPTION OF THE PREFERRED EMBODIMENTS

It has been discovered, surprisingly and unexpectedly, that certain acrylic tetrapolymers meet this need. In particular, these polymers not only make it possible to tighten the skin effectively and so to mask wrinkles and fine lines and signs of tiredness, etc., but also have self-emulsification properties, which allow them to be formulated in emulsion without need for a surfactant, and so make it possible to obtain, readily, compositions which are stable and have a satisfactory tightening effect.

These polymers have already been described as being useful for enhancing the water resistance and/or abrasion resistance of compositions (US 2003/0021847) such as sun compositions (WO 03/082237) and for preventing their fluffing (U.S. application Ser. No. 10/372,330). It has also been suggested that they might prevent the skin drying out and possess anti-inflammatory properties, by virtue of their capacity to structure the epidermal lipids and the sebum and so to reinforce the lipid barrier in the stratum corneum (WO 03/103615).

However, to the best of present knowledge, it has never yet been suggested that these polymers have the capacity to tighten the skin and thus be useful for, e.g., smoothing wrinkles.

The present invention accordingly provides a (cosmetic treatment) process for treating human skin, particularly human skin in need thereof such as wrinkled skin, skin showing signs of tiredness, and skin in need of treatment with a tightening agent, comprising the topical application to said skin of a composition comprising, in a physiologically acceptable medium, at least one tetrapolymer of methacrylic acid, methyl methacrylate, butyl acrylate and C₁₆-C₂₀ alkyl (meth)acrylates, said composition preferably being devoid of sunscreen agents.

The invention also provides for the (cosmetic) use of at least one tetrapolymer of methacrylic acid, methyl methacrylate, butyl acrylate and C₁₆-C₂₀ alkyl (meth)acrylates as a tightening agent for the human skin, especially for the purpose of smoothing out wrinkles and/or disguising the signs of tiredness on the skin.

The tetrapolymer used according to the invention preferably comprises from 0.5% to 10% by weight, advantageously from 1% to 2% by weight, of methacrylic acid; from 25% to 90% by weight, advantageously from 10% to 40% by weight, of methyl methacrylate; from 10% to 90% by weight, advantageously from 30% to 50% by weight, of butyl acrylate; and from 25% to 75% by weight, advantageously from 25% to 35% by weight, of C₁₆-C₂₀ alkyl (meth)acrylates. By “C₁₆-C₂₀ alkyl (meth)acrylates” are meant one or more monomers composed of alkyl (meth)acrylate whose alkyl chain contains 16 to 20 carbon atoms, preferably a mixture of monomers composed of C₁₆, C₁₈ and C₂₀ alkyl (meth)acrylates. The alkyl (meth)acrylates preferably include from 25% to 35% by weight of cetyl methacrylate, from 55% to 65% by weight of stearyl acrylate and from 5% to 15% by weight of eicosyl acrylate.

Tetrapolymers as defined above and also the process for preparing them are described in particular in application U.S. 2003/0021847.

They can be prepared by emulsion polymerization of the above monomers in the presence of a free-radical initiator such as hydrogen peroxide, tert-butyl hydroperoxide or sodium, potassium, lithium or ammonium persulphate, the initiator being optionally combined with a reducing agent, so as to form a redox system, and with a catalyst composed of a transition metal such as a salt of copper or of iron. The reaction may for example be carried out at a temperature of between 10 and 120° C., preferably in the region of 85° C., for an approximate time of three hours.

A tetrapolymer of this type is in particular available from the company Rohm & Haas under the trade name Allianz OPT in the form of an aqueous-glycolic dispersion containing 48% active substance.

The amount of tetrapolymer incorporated in the composition according to the invention may vary within a wide range in accordance with the desired effect. To provide an order of magnitude, the composition may include from 0.1% to 10% by weight, and preferably from 0.2% to 5% by weight, of tetrapolymer, relative to the total weight of the composition. Of course, the amount of tetrapolymer is preferably an amount effective to provide the intended benefit, for example, to smooth out wrinkles, treat the signs of tired skin, provide a tightening effect, etc. Such amounts may fall within the above guidelines, and in any event, is easily determinable by one of ordinary skill in the art based on the present disclosure.

This composition is generally preferably adapted to topical application to the skin and therefore generally comprises a physiologically acceptable medium, in other words a medium which is compatible with the skin and/or its epidermal derivatives. The medium is preferably a cosmetically acceptable medium, which is to say that it has a color, odor and feel which are pleasant and that it does not produce unacceptable feelings of discomfort (tingling, pulling, redness) likely to dissuade the consumer from using this composition.

This is particularly important when the composition according to the invention is intended for application around the eyes, which constitutes one advantageous embodiment of the present invention.

Naturally, the invention compositions are preferably applied in a knowing manner, and for a known purpose, to skin in need of the intended benefits of the invention.

The composition according to the invention may additionally be presented in any form, including any of the forms of formulation that are conventionally used for topical application, and particularly in the form of dispersions of the lotion or aqueous gel type, emulsions with a liquid or semi-liquid consistency, of the milk type, which are obtained by dispersing a fatty phase in an aqueous phase (H/W) or vice versa (W/O), or suspensions or emulsions of soft, semi-solid or solid consistency, of the cream or gel type, or else multiple emulsions (W/O/W or O/W/O), microemulsions, vesicular dispersions of ionic and/or nonionic type, or wax/aqueous phase dispersions. These compositions are prepared according to the usual methods.

According to one preferred embodiment of the invention the composition is presented in the form of an O/W emulsion.

Oils which can be used in the composition according to the invention include the following:
hydrocarbon oils of animal origin, such as perhydrosqualene;
hydrocarbon oils of plant origin, such as liquid triglycerides of fatty acids containing 4 to 10 carbon atoms or else, for example, vegetable oils such as apricot kernel oil and karite butter oil;
synthetic esters and ethers, especially those of fatty acids, such as the oils of formulae R¹COOR² and R¹OR² in which R¹ represents the residue of a fatty acid containing 8 to 29 carbon atoms and R² represents a branched or unbranched hydrocarbon chain containing 3 to 30 carbon atoms;
linear or branched hydrocarbons, mineral or synthetic in origin, such as volatile or non-volatile liquid paraffins and derivatives thereof, isohexadecane, isododecane, petroleum jelly, polydecenes, and hydrogenated polyisobutene such as Parleam® oil;
natural or synthetic essential oils;
fatty alcohols having 8 to 26 carbon atoms, such as cetyl alcohol, stearyl alcohol and a mixture thereof (cetyl stearyl alcohol) or octyldodecanol;
fluoro oils partially treated with hydrocarbons and/or silicones, such as those described in document JP-A-2-295912;
silicone oils, such as volatile or non-volatile polydimethylsiloxanes (PDMS) containing a linear or cyclic silicone chain which are liquid or pasty at ambient temperature, especially cyclopolydimethylsiloxanes (cyclomethicones) such as cyclohexasiloxane and cyclopentasiloxane; polydimethylsiloxanes containing alkyl, alkoxy or phenyl groups pendently or at the end of a silicone chain, said groups having 2 to 24 carbon atoms; phenyl silicones such as phenyltrimethicones, phenyldimethicones, phenyltrimethylsiloxydiphenylsiloxanes, diphenyldimethicones, diphenylmethyldiphenyltrisiloxanes, 2-phenylethyl trimethylsiloxysilicates and polymethylphenylsiloxanes; and
mixtures thereof.

The other fatty substances which may be present in the oily phase are, for example, fatty acids containing 8 to 30 carbon atoms, such as stearic acid, lauric acid, palmitic acid and oleic acid; waxes such as lanoline, beeswax, carnauba wax or candelilla wax; and gums such as silicone gums (dimethiconol).

These fatty substances may be selected variously by the skilled person so as to prepare a composition having the desired properties of, for example, consistency or texture.

This composition may further comprise various adjuvants which are commonly used in the field of cosmetology, such as fillers, preservatives, sequestrants, dyes and fragrances.

Fillers that may be mentioned include, for example, particles of polyamide (Nylon) in spherical form or in microfibre form; microspheres of polymethyl methacrylate; powders of ethylene-acrylate copolymer; expanded powders such as hollow microspheres and in particular the microspheres formed from a terpolymer of vinylidene chloride, acrylonitrile and methacrylate, which are sold under the name Expancel by the company Kemanord Plast; powders of natural organic materials, such as crosslinked or non-crosslinked starch powders, especially maize starch, wheat starch or rice starch powders, such as starch powders crosslinked with octenylsuccinic anhydride; silicone resin microbeads such as those sold under the name Tospearl by the company Toshiba Silicone; silica; metal oxides such as titanium dioxide or zinc oxide; mica; and mixtures thereof.

The skilled person will of course take care to ensure that this or these additional compounds, where appropriate, and/or the amount thereof are selected such that the advantageous properties of the composition according to the invention are not, or not substantially, adversely affected by the intended addition.

In particular, according to one advantageous embodiment, the composition used according to the invention includes no, or substantially no, surfactant, in other words less than 1% by weight, or even less than 0.5% by weight, of surfactant, relative to the total weight of the composition. By surfactant is meant any compound or mixture of compounds identified as such in at least one of the following two works: McCutcheon's: Emulsifiers and Detergents, International Edition; and International Cosmetic Ingredient Dictionary and Handbook (CTFA).

This is because it has been demonstrated that the tetrapolymer according to the invention allows stable compositions to be realized without the need to employ a surfactant or even thickeners.

However, in order to enhance the stability of such compositions, especially when the tetrapolymer is present only at a level of 4% by weight or less (of active substance) in the composition, it may be useful to combine it with at least one thickener, which for example may be selected from the homopolymers and copolymers of acrylic acid (such as carbomers and the copolymers sold under the names Pemulen TR1 and Pemulen TR2 by Noveon) and the homopolymers and copolymers of acrylamide and/or of 2-acrylamido-2-methylpropanesulphonic acid (AMPS), such as those sold under the names Sepigel 305 and Simulgel 600 by Seppic and under the names Hostacerin AMPS and Aristoflex HMS by Clariant.

Moreover, in order to reinforce the anti-ageing effects of the composition used according to the invention, it may be advantageous to introduce into said composition at least one compound selected from desquamating agents; moisturizers; depigmenting or propigmenting agents; anti-glycation agents; NO-synthase inhibitors; agents which stimulate the synthesis and/or prevent the degradation of dermal or epidermal macromolecules; agents which stimulate the proliferation of fibroblasts and/or keratinocytes or stimulate the differentiation of keratinocytes; skin-decontracting agents and/or muscle relaxants; tightening agents; anti-pollution agents and/or free-radical scavengers; agents which act on the microcirculation; agents which act on the energy metabolism of the cells; and mixtures thereof.

Examples of such addition compounds are retinol and its derivatives, such as retinyl palmitate; ascorbic acid and its derivatives, such as magnesium ascorbyl phosphate and ascorbyl glucoside; tocopherol and its derivatives, such as tocopheryl acetate; nicotinic acid and its precursors, such as nicotinamide; ubiquinone; glutathione and its precursors, such as L-2-oxothiazolidine-4-carboxylic acid; plant extracts and especially plant proteins and their hydrolysates, and also phytohormones; marine extracts, such as algal extracts; bacterial extracts; sapogenins, such as diosgenin and wild yam extracts containing it; ceramides; α-hydroxy acids; β-hydroxy acids, such as salicylic acid and n-octanoyl-5-salicylic acid; resveratrol; oligopeptides and pseudodipeptides and their acyl derivatives; manganese salts and magnesium salts, particularly the gluconates; and mixtures thereof.

The invention will now be illustrated by the following, non-limiting examples. In these examples the amounts are indicated as percentages by weight, unless indicated otherwise.

### EXAMPLES

### Example 1

### Demonstration of the Tightening Effect In Vivo

An O/W emulsion was prepared which had the composition below, in a manner conventional for the skilled person.

| | |
|---|---|
| Glycerol | 5% |
| Preservatives | 0.4% |
| Acrylic copolymer⁽¹⁾ | 1% |
| Neutralizing agent | 0.05% |
| Aqueous dispersion of acrylic | |
| tetrapolymer⁽²⁾ | 2% |
| AMPS copolymer⁽³⁾ | 0.3% |
| Hydrogenated polyisobutene | 3% |
| Cyclohexasiloxane | 4% |
| Isohexadecane | 3% |
| Fragrance | qs |
| Water | qs 100% |

⁽¹⁾Synthalen W2000 from Sigma (acrylate/palmeth-25 acrylate copolymer)
⁽²⁾Allianz OPT from Rohm & Haas (48% active substance)
⁽³⁾Aristoflex HMS from Clariant (crosslinked AMPS/steareth-25 methacrylate copolymer)

This composition was applied to a panel of five women who had wrinkles and fine lines. It was applied precisely to half of the face (and the area surrounding the corresponding eye) and the condition of the skin was evaluated immediately following application, by comparison with the other half of the face (untreated).

Three out of five women judged that this composition produced a moderate or slight smoothing effect on the face, which appeared smoother and more relaxed, and a moderate or slight tightening effect around the eye, which lead to the disguising of the fine lines.

It was verified in vitro, moreover, that the AMPS copolymer and the acrylic copolymer had no perceptible tightening effect at the levels indicated in the formula above.

This example therefore demonstrates the tightening power of the tetrapolymer used according to the invention.

### Example 2

### Demonstration of the Tightening Effect In Vivo

The tightening effect of two acrylic polymers was evaluated in vitro, using a 0.96% active substance solution, and was set against that of the tetrapolymer according to the invention, which was assigned a value of 100. The results are presented in Table 1 below.

TABLE 1

| | Tightening |
|---|---|
| Acrylic polymer | effect |
| Acrylic tetrapolymer according to the invention (Allianz | 100% |
| OPT from Rohm & Haas) | |
| Acrylic acid/ethyl acrylate copolymer (Aculyn 33 from | 94% |
| Rohm & Haas) | |
| Acrylic polymer (Carbopol Aqua SF1 from Noveon) | 83% |

These results show that the tetrapolymer according to the invention exhibits better skin tightening properties than other acrylic polymers.

### Example 3

### Demonstration of the Stability of Emulsions

Three O/W emulsions were prepared, denoted below by emulsions A, B and C, having the composition below, in which three different acrylic polymers were incorporated respectively.

| | |
|---|---|
| Alcohol | 10% |
| Hydrogenated polyisobutene | 10% |
| Glycerol | 5% |
| Cyclohexasiloxane | 5% |
| Acrylic polymer | 4.6% (active substance) |
| Preservatives | 0.4% |
| Fragrance | qs |
| Water | qs 100% |

These compositions were prepared as follows: the hydrogenated polyisobutene and the cyclohexasiloxane were dispersed at ambient temperature, in the mixer, in the mixture of water, acrylic polymer, glycerol and preservatives, and then the alcohol and the fragrance were added at the same temperature.

The stability of these emulsions was evaluated with the naked eye. The results are compiled in Table 2 below.

TABLE 2

| Emulsion | Acrylic polymer | Stability | Cosmetic result |
|---|---|---|---|
| A | Acrylic tetrapolymer | Formula with a | Does not fluff up on |
| | according to the invention | homogeneous | application. Fresh, |
| | (Allianz OPT from Rohm | appearance, highly | glides on. Rich and |
| | & Haas) | fluid milk. No phase | film-forming at the |
| | | separation. | end. |
| B | Acrylic acid/ethyl acrylate | Salting-out. | Impossible to |
| | copolymer (Aculyn 33 | Accumulation of | evaluate (emulsion |
| | from Rohm & Haas) | gelled fatty phase at | showed phase |
| | | the surface. | separation) |
| C | Acrylic polymer (Carbopol | Formula showing | Impossible to |
| | Aqua SF1 from Noveon) | phase separation; | evaluate (emulsion |
| | | heterogeneous mixture | showed phase |
| | | comprising a gelled | separation) |
| | | fatty phase at the | |
| | | surface. Substantial | |
| | | salting-out. | |

These results show that the tetrapolymer according to the invention allows stable emulsions to be formulated without surfactant. Given that surfactants are not always well tolerated by highly sensitive skins, this tetrapolymer is therefore well adapted to use on sensitive skins which present signs of ageing and/or tiredness.

Furthermore, the possibility of formulating the tetrapolymer according to the invention without surfactant allows it to be formulated with a minimum of constraints in very varied cosmetic vehicles, since there will be no need to take account of the chemical incompatibilities between certain surfactants and other constituents of these vehicles. This tetrapolymer therefore offers a large latitude in formulation.

The above written description of the invention provides a manner and process of making and using it such that any person skilled in this art is enabled to make and use the same, this enablement being provided in particular for the subject matter of the appended claims, which make up a part of the original description and including a process for treating wrinkled human skin, comprising the topical application to said skin of a composition comprising, in a physiologically acceptable medium, a tetrapolymer of methacrylic acid, methyl methacrylate, butyl acrylate and C₁₆-C₂₀ alkyl (meth)acrylates, said composition preferably being devoid of sunscreen agents. Also fully enabled herein is the use of a tetrapolymer of methacrylic acid, methyl methacrylate, butyl acrylate and C₁₆-C₂₀ alkyl (meth)acrylates as a tightening agent for human skin. Particular uses of the invention fully described and enabled also include the use of the invention tetrapolymer for smoothing out wrinkles and/or disguising the signs of tiredness on the skin.

As used herein, the phrases “selected from the group consisting of,” “chosen from,” “selected from,” and the like include mixtures of the specified materials.

Where compounds are described as, e.g, “retinol and its derivatives” an alternate is “retinol and retinol compounds” where retinol derivatives and compounds share a common core/structure with retinol. One of ordinary skill in the art knows how to identify derivatives and compounds of compound X based on the structure of X and the similarity in structure of the derivatives and compounds.

Where a term is enclosed by parentheses it is an optional modifier. For example, the term “(cosmetic treatment) process” describes both a process in general and a cosmetic treatment process.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for treating wrinkled human skin, comprising topically applying to skin in need thereof a composition comprising, in a physiologically acceptable medium, a wrinkle treating effective amount of at least one tetrapolymer of methacrylic acid, methyl methacrylate, butyl acrylate and C₁₆-C₂₀ alkyl (meth)acrylates, said composition being devoid of sunscreen agents.

2. The process according to claim 1, wherein said tetrapolymer comprises 1%-2% by weight of methacrylic acid, 10%-40% by weight of methyl methacrylate, 30%-50% by weight of butyl acrylate and 25%-35% by weight of C₁₆-C₂₀ alkyl (meth)acrylates.

3. The process according to claim 1, wherein the alkyl (meth)acrylates comprise 25%-35% by weight of cetyl methacrylate, 55%-65% by weight of stearyl acrylate and 5%-15% by weight of eicosyl acrylate.

4. The process according to claim 1, wherein said composition is applied around the eyes.

5. The process according to claim 1, wherein said composition comprises 0.1%-10% by weight of tetrapolymer, relative to the total weight of the composition.

6. The process according to claim 5, wherein the composition comprises 0.2%-5% by weight of tetrapolymer, relative to the total weight of the composition.

7. The process according to claim 1, wherein the composition comprises less than 1% by weight of surfactant, relative to the total weight of the composition.

8. The process according to claim 7, wherein the composition comprises less than 0.5% of surfactant.

9. The process according to claim 7, wherein the composition further comprises at least one thickener selected from homopolymers and copolymers of acrylic acid, homopolymers and copolymers of acrylamide and/or of 2-acrylamido-2-methylpropanesulphonic acid, and mixtures thereof.

10. The process according to claim 1, wherein the composition further comprises at least one compound selected from desquamating agents; moisturizers; depigmenting or propigmenting agents; anti-glycation agents; NO-synthase inhibitors; agents which stimulate the synthesis and/or prevent the degradation of dermal or epidermal macromolecules; agents which stimulate the proliferation of fibroblasts and/or keratinocytes or stimulate the differentiation of keratinocytes; skin-decontracting agents and/or muscle relaxants; other tightening agents; anti-pollution agents and/or free-radical scavengers; agents which act on the microcirculation; agents which act on the energy metabolism of the cells; and mixtures thereof.

11. The process according to claim 1, wherein the composition further comprises at least one compound selected from retinol and its derivatives; ascorbic acid and its derivatives; tocopherol and its derivatives; nicotinic acid and its precursors; ubiquinone; glutathione and its precursors; plant extracts; plant proteins and their hydrolysates; phytohormones; marine extracts; bacterial extracts; sapogenins; ceramides; α-hydroxy acids; β-hydroxy acids; resveratrol; oligopeptides and pseudodipeptides and their acyl derivatives; manganese salts; magnesium salts; and mixtures thereof.

12. A process, comprising topically applying to human skin in need of being tightened a composition comprising, in a physiologically acceptable medium, a skin tightening effective amount of at least one tetrapolymer of methacrylic acid, methyl methacrylate, butyl acrylate and C₁₆-C₂₀ alkyl (meth)acrylates, said composition being devoid of sunscreen agents.

13. The process according to claim 12, wherein said tetrapolymer comprises from 1%-2% by weight of methacrylic acid, 10%-40% by weight of methyl methacrylate, 30%-50% by weight of butyl acrylate and 25%-35% by weight of C₁₆-C₂₀ alkyl (meth)acrylates.

14. The process according to claim 12, wherein the alkyl (meth)acrylates comprise 25%-35% by weight of cetyl methacrylate, 55%-65% by weight of stearyl acrylate and 5%-15% by weight of eicosyl acrylate.

15. The process according to any one of claim 12, wherein said composition is applied around the eyes.

16. The process according to any one of claim 12, wherein said composition comprises 0.1%-10% by weight of tetrapolymer, relative to the total weight of the composition.

17. The process according to claim 16, wherein the composition comprises 0.2%-5% by weight of tetrapolymer, relative to the total weight of the composition.

18. The process according to claim 12, wherein the composition comprises less than 1% by weight of surfactant, relative to the total weight of the composition.

19. The process according to claim 18, wherein the composition comprises less than 0.5% of surfactant.
